(19) United States
(12) Patent Application Publication
(10) Pub. No.: US 2019/0051212 A1
(43) Pub. Date: Feb. 14, 2019

(54) METHOD AND SYSTEM FOR SUGGESTING NUTRIENT INTAKE

(71) Applicant: National Taiwan Normal University, Taipei City (TW)

(72) Inventors: Jon-Chao HONG, Taipei City (TW); Kai-Hsiang YANG, Taipei City (TW)

(73) Assignee: National Taiwan Normal University, Taipei City (TW)

(21) Appl. No.: 15/676,379

(22) Filed: Aug. 14, 2017

(51) Int. Cl. G09B 19/00; G09B 5/02; G09B 5/04; A61B 5/00

(52) U.S. Cl. CPC G09B 19/0092; G09B 5/02; G09B 5/04; A61B 5/486; A61B 5/4866; A61B 5/7267; A61B 5/0022

(57) ABSTRACT

A method for suggesting nutrient intake includes: by a statistical platform, the steps of obtaining a training sample, building a classification model based on physical data, nutrient intake data and effect data of the training sample thus obtained, and providing the classification model to a management platform; and by the management platform, a step of when receiving a to-be-classified sample that includes physical data from a client device, determining suggested nutrient intake data by inputting the physical data of the to-be-classified sample into the classification model to obtain an output of the classification model which serves as the suggested nutrient intake data.

### FIELD

The disclosure relates to a method and a system for processing data by cooperating with a cloud database, and more particularly to a method and a system for suggesting nutrient intake by cloud computing based on numerical data from at least one client device.

### BACKGROUND

Many people have trouble determining the amount of nutrients to consume, i.e., recommended nutrient intake, and such situation may be further complicated in view of individual differences, such as differences in personal physical features (e.g., body weight, body height, etc.) and physiological conditions (e.g., variations in blood sugar levels).

As a result, there is a demand for a way to determine the recommended nutrient intake, taken into consideration various personal physical features and physiological conditions.

### SUMMARY

Therefore, an object of the disclosure is to provide a method and a system for suggesting nutrient intake.

According to one aspect of the disclosure, the system is adapted to cooperate with a client device and includes a statistical platform and a management platform.

The statistical platform is configured to obtain a training sample that includes physical data which is associated with a physical feature of a consumer, nutrient intake data which is associated with an amount of a nutrient consumed by the consumer, and effect data which is associated with an effect on a physiological parameter of the consumer after the consumer has consumed the amount of the nutrient, to build a classification model based on the physical data, the nutrient intake data and the effect data of the training sample thus obtained, and to provide the classification model that maps the physical data to the nutrient intake data which results in the effect on the physiological parameter.

The management platform is configured to obtain the classification model, and to determine, when receiving a to-be-classified sample that includes physical data from the client device, suggested nutrient intake data which is associated with a suggested amount of the nutrient for the to-be-classified sample by inputting the physical data of the to-be-classified sample into the classification model to obtain an output of the classification model which serves as the suggested nutrient intake data.

According to another aspect of the disclosure, the method is to be implemented by the system and the client device that are mentioned above. The method includes the steps of:
a) obtaining, by the statistical platform, a training sample that includes physical data which is associated with a physical feature of a consumer, nutrient intake data which is associated with an amount of a nutrient consumed by the consumer, and effect data which is associated with an effect on a physiological parameter of the consumer after the consumer has consumed the amount of the nutrient;
b) building, by the statistical platform, a classification model based on the physical data, the nutrient intake data and the effect data of the training sample thus obtained, and providing, by the statistical platform, the classification model to the management platform, the classification model mapping the physical data to the nutrient intake data associated with the amount of the nutrient which when consumed results in the effect on the physiological parameter; and
c) determining, by the management platform when receiving a to-be-classified sample that includes physical data from the client device, suggested nutrient intake data which is associated with a suggested amount of the nutrient for the to-be-classified sample by inputting the physical data of the to-be-classified sample into the classification model to obtain an output of the classification model which serves as the suggested nutrient intake data.

### BRIEF DESCRIPTION OF THE DRAWINGS

Other features and advantages of the disclosure will become apparent in the following detailed description of the embodiment with reference to the accompanying drawings, of which:

FIG. 1 is a block diagram illustrating an embodiment of a system for suggesting nutrient intake according to this disclosure;

FIG. 2 is a flow diagram illustrating an embodiment of a method for suggesting nutrient intake according to this disclosure; and

FIG. 3 is a flow diagram illustrating an embodiment of steps in an update phase of the method according to this disclosure.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

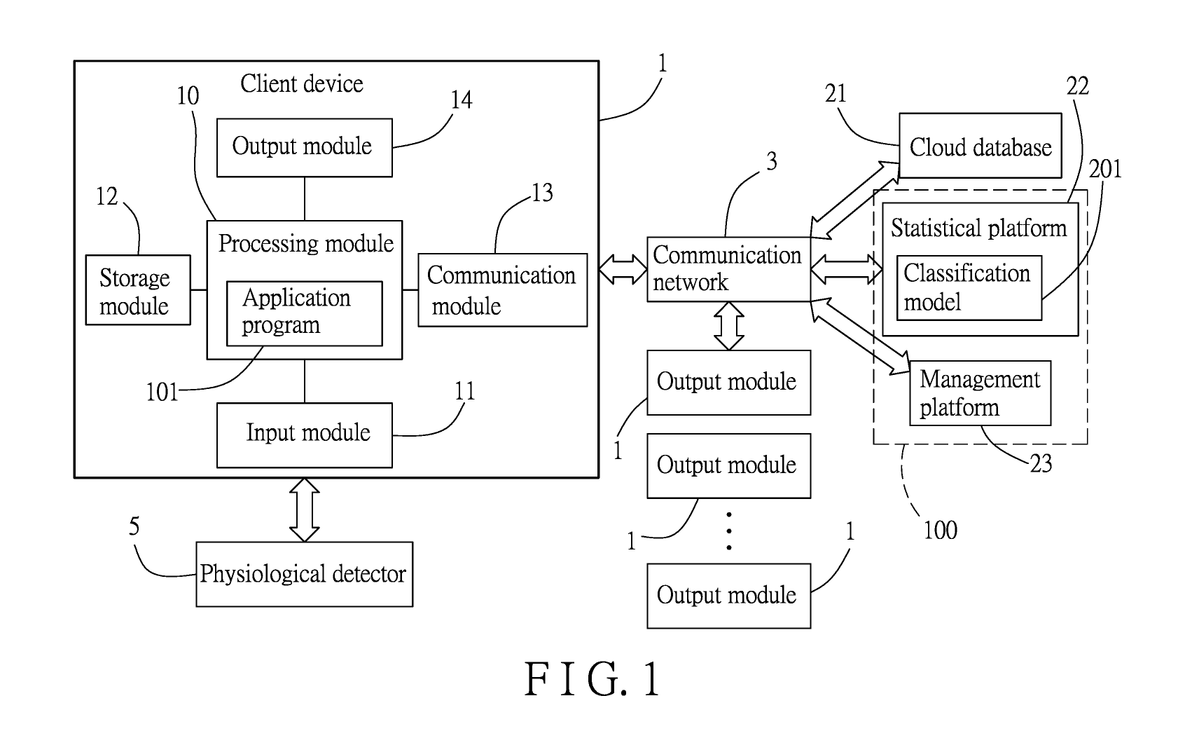

FIG. 1 illustrates an embodiment of a system 100 for suggesting nutrient intake according to the disclosure. The system 100 is adapted to cooperate with a plurality of client devices 1 and a cloud database 21. The system 100 includes a statistical platform 22 and a management platform 23. The client devices 1, the cloud database 21, the statistical platform 22 and the management platform 23 are communicable with each other via a communication network 3.

The statistical platform 22 is configured to obtain a plurality of training samples from the cloud database 21. Each of the training samples includes at least one entry of physical data which is associated with a physical feature of a consumer, at least one entry of nutrient intake data which is associated with an amount of a nutrient consumed by the consumer, and at least one entry of effect data which is associated with an effect on a physiological parameter of the consumer after the consumer has consumed the amount of the nutrient with which the at least one entry of nutrient intake data is associated. In a training phase of the system 100, the statistical platform 22 is configured to build a classification model 201 based on the physical data, the nutrient intake data and the effect data of the training samples thus obtained. Thereafter, the statistical platform 22 is configured to provide the management platform 23 with the classification model 201 that maps the physical data to the nutrient intake data associated with the amount of the nutrient which when consumed results in the effect on the physiological parameter. The training samples may be acquired by the cloud database 21 from published information on websites (e.g., social networking websites) uploaded by any one of the client devices 1 via the Internet. Alternatively, the training samples may be directly obtained from one(s) of the client devices 1 by the cloud database 21.

It should be noted that, for the sake of clearer explanation, only one entry of physical data is given for each of the training samples as an example hereinafter. However, in practice, multiple entries of physical data respectively associated with multiple physical features may be provided in a single training sample. Similarly, one entry of nutrient intake data and one entry of effect data are recited hereinafter for explanatory purposes, but do not place a limitation on the quantity of data entries in a single training sample. Moreover, the term “the physical data” mentioned in this disclosure refers to one or more entries of physical data, the term “the nutrient intake data” mentioned in this disclosure refers to one or more entries of nutrient intake data, and the term “the effect data” mentioned in this disclosure refers to one or more entries of effect data.

In some embodiments, the physical feature includes at least one of a Body Mass Index (BMI), a waist circumference, age, body weight, body height, or a special physical factor (e.g., hypertension, hypercholesterolemia, hyperlipidemia, etc.). Accordingly, the physical data may be a BMI value, a length of waist circumference, a number of years of age, a body weight value, a body height value, or a description of the specific physical factor. The nutrient intake data includes at least one of an amount of sugar, an amount of salt, an amount of lipid, or an amount of any nutrient utilized to maintain life functions. The effect data is implemented to include at least one effect on a physiological parameter of a consumer after the consumer consumes a certain amount of the nutrient, and the effect on the physiological parameter may be represented quantitatively (e.g., indicated by variations in blood sugar levels after the consumer consumes one gram of sugar, or the absence thereof) or qualitatively (e.g., indicated as being normal or abnormal in blood sugar levels after the consumer consumes one gram of sugar).

In this embodiment, the classification model 201 is implemented by vector space model (VSM) and similarity measurement so as to realize statistical classification, and is utilized in an application phase of the system 100 to determine suggested nutrient intake data, which is associated with a suggested amount of the nutrient, for a to-be-classified sample.

It is noted herein that for the purpose of establishing a classification model 201 that is suitable for recommending a nutrient intake amount to a user (to result in desirable, beneficial effects), it would be desirable for each training sample used in the training phase of the system 100 to include effect data that is associated with a beneficial or desirable effect, and not adverse effect on the physiological parameter. An effect of maintaining a current blood sugar level (i.e., no variation in blood sugar level after consumption of the nutrient) may in many cases be desirable and considered beneficial.

The to-be-classified sample includes physical data associated with a physical feature of a user of one of the client devices 1, and is received by the management platform 23 from said one of the client devices 1. For example, the physical data may indicate that the user is 65 kg in terms of body weight (the physical feature). The goal is to make the suggested amount of the nutrient suitable for the user, the specific physical feature of whom is of the specific value or condition (e.g., a user that is 65 kg in terms of body weight). Through the classification model 201, the suggested nutrient intake data for the to-be-classified sample is determined based on the nutrient intake data of the training samples that corresponds to the physical data which is within a predetermined range of similarity relative to the physical data of the to-be-classified sample, and that is associated with the amount of the nutrient which when consumed results in a desirable effect on the physiological parameter, e.g., to keep a value of the physiological parameter in a reasonable range. However, it should be noted that implementation of the classification model 201 may vary in other embodiments and is not limited to what are disclosed herein.

In the application phase of the system 100, the management platform 23 is configured to obtain the classification model 201 from the statistical platform 22, and to determine, when receiving the to-be-classified sample, which includes the physical data, from said one of the client devices 1, the suggested nutrient intake data by inputting the physical data of the to-be-classified sample into the classification model 201 so as to obtain an output of the classification model 201 which serves as the suggested nutrient intake data. In a variation of this embodiment, given an entry of physical data, e.g., associated with the physical feature of body weight (or body height), of a to-be-classified sample, the suggested nutrient intake data for the to-be-classified sample may be determined as an average of multiple entries of the nutrient intake data which correspond respectively to multiple entries of the physical data that are within the predetermined range of similarity relative to the physical data of the to-be-classified sample in terms of the same physical feature (body weight (or body height)), and which are associated respectively with the amounts of the nutrient which when consumed results in beneficial or desirable effects on the physiological response.

Referring to FIG. 1 again, each of the client devices 1 includes a processing module 10, an input module 11, a storage module 12, a communication module 13 and an output module 14. The processing module 10 is electrically connected to the input module 11, the storage module 12, the communication module 13 and the output module 14. The processing module 10 stores an application program 101. Each of the client devices 1 is communicable with the management platform 23 via the communication module 13 thereof and the communication network 3. The input module 11 serves as an interface for user's input of the physical data of the to-be-classified sample to the application program 101. The application program 101 is executed such that the processing module 10 is configured to transmit, to the management platform 23 of the system 100 via the communication module 13 and the communication network 3, the physical data of the to-be-classified sample thus inputted, to serve as an input to the classification model 201, so as to obtain, via the communication module 13 and the communication network 3, the suggested nutrient intake data outputted by the classification model 201. The storage module 12 is configured to record the physical data of the to-be-classified sample inputted via the input module 11 and the suggested nutrient intake data outputted by the classification model 201. The output module 14 is configured to provide an indication of the suggested nutrient intake data for informing the user.

In this embodiment, each of the client devices 1 may be an electronic device that can access the Internet, such as a smartphone, a personal computer, or the like, but is not limited thereto. The processing module 10, the input module 11, the storage module 12, the communication module 13 and the output module 14 may be implemented by electronic components ordinarily disposed in or connected to a smartphone, a personal computer, or the like, and configurable/programmable to carry out the aforementioned operations.

In some embodiments, the cloud database 21, the statistical platform 22 and the management platform 23 may be implemented separately, and each may be one of a server computer, a database server, a computing server, and the like, but is not limited thereto.

In some embodiments, the cloud database 21, the statistical platform 22 and the management platform 23 may be implemented to be integrated as one of a server computer, a database server, a computing server, and the like, but implementation is not limited to such.

In this embodiment, the processing module 10 may be implemented by at least one of a central processing unit (CPU), a microcontroller unit (MCU), or a System on Chip (SoC), but is not limited thereto.

In this embodiment, the input module 11 may be implemented by at least one of a keyboard, a touchscreen, a pointing device, or an audio input device, but is not limited thereto.

In this embodiment, the storage module 12 may be implemented by at least one of a flash memory, a hard disk drive (HDD) or a solid state disk (SSD), but is not limited thereto.

In this embodiment, the communication module 13 may be implemented by at least one of a wired/wireless network interface (e.g., a Bluetooth interface or a Wi-Fi interface), but is not limited thereto.

In this embodiment, the output module 14 may be implemented by at least one of a display, such as a liquid-crystal display (LCD), a speaker, or a printer, but is not limited thereto.

To improve classification performance of the classification model 201, i.e., to improve accuracy of the suggested nutrient intake data so that a suggested amount of the nutrient may be more appropriate for a next to-be-classified sample, each of the client devices 1 further cooperates with a physiological detector 5. The management platform 23 is further configured to obtain, from one of the client devices 1, plural entries of nutrient intake data each of which is associated with an amount of the nutrient consumed by the user of the one of the client devices 1 as previously suggested by a corresponding entry of suggested nutrient intake data, and which correspond respectively to a sequence of time points. Also, the management platform 23 is further configured to obtain, from the one of the client devices 1, plural entries of physiological data, each of which is associated with an effect on a physiological parameter of the user after a respective one of the sequence of time points at which the user consumes the amount of the nutrient as suggested by a corresponding entry of the suggested nutrient intake data, and which are obtained by the one of the client devices 1 via the corresponding physiological detector 5. After obtaining the plural entries of nutrient intake data and the plural entries of corresponding physiological data, the management platform 23 is configured to record the plural entries of nutrient intake data indexed respectively by the sequence of time points in an account corresponding to the one of the client devices 1 in the cloud database 21, and to record the plural entries of physiological data indexed respectively by the sequence of time points in the account in the cloud database 21.

Therefore, a single account can be used on different client devices for each user, and a service of nutrient intake suggestion can be conveniently provided for the account when the plural entries of physiological data corresponding thereto are recorded in the cloud database 21. Thereafter, the management platform 23 is further configured to update (e.g., refine) the classification model 201 based on the physical data of the to-be-classified sample which corresponds to the user of the one of the client devices 1, on the plural entries of nutrient intake data and on the plural entries of physiological data.

In this embodiment, the physiological detector 5 may be implemented to include one or more of a thermometer, a respiration sensor, a skin impedance sensor, an electromyograph, an electrocardiograph and a pulse meter, but is not limited thereto.

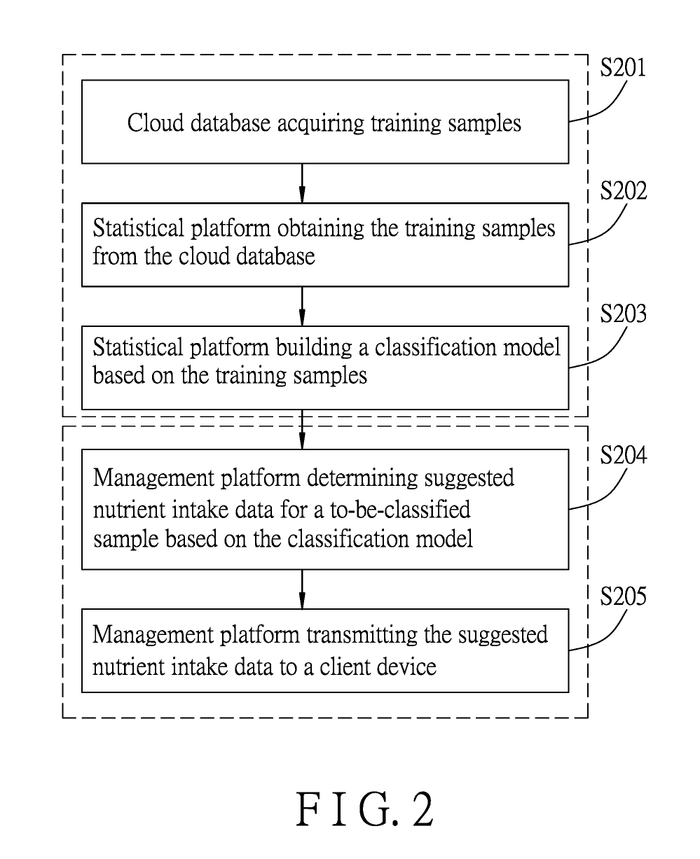

An embodiment of a method for suggesting nutrient intake according to this disclosure will now be described with reference to FIGS. 1 and 2. The method is to be implemented by the system 100, the cloud database 21, the client devices 1 and the physiological detectors 5 that are previously mentioned. Each of the client devices 1 stores the application program 101. The method includes the following steps (S201-205), wherein steps (S201-203) are performed in the training phase and steps (S204-S205) are performed in the application phase.

In step (S201), the cloud database 21 acquires a plurality of training samples from published information on the websites, such as social networking websites, uploaded by at least one of the client devices 1 via the Internet. Alternatively, the cloud database 21 may acquire the training samples directly from the client devices 1.

In step (S202), the statistical platform 22 obtains the plurality of training samples from the cloud database 21. Each of the training samples includes at least one entry of physical data which is associated with a physical feature of a consumer, at least one entry of nutrient intake data which is associated with an amount of a nutrient consumed by the consumer, and at least one entry of effect data which is associated with a physiological response of the consumer after the consumer has consumed the amount of the nutrient with which the entry of nutrient intake data is associated.

In step (S203), the statistical platform 22 builds a classification model 201 based on the physical data, the nutrient intake data and the effect data of the training samples thus obtained. The statistical platform 22 provides the classification model 201 thus built to the management platform 23. The classification model 201 maps the physical data to the nutrient intake data associated with the amount of the nutrient which when consumed results in the effect on the physiological response.

In step (S204), when receiving a to-be-classified sample that includes physical data from one of the client devices 1, the management platform 23 determines suggested nutrient intake data which is associated with a suggested amount of the nutrient for the to-be-classified sample by inputting the physical data of the to-be-classified sample into the classification model 201 to obtain an output of the classification model 201 which serves as the suggested nutrient intake data.

In step (S205), the management platform 23 transmits the output of the classification model 201, which serves as the suggested nutrient intake data, to said one of the client devices 1 from which the to-be-classified sample is received.

A scenario where a male user who is 176 cm tall and weighs 75 kg wishes to inquire a suggested amount of sugar so as to maintain the blood sugar level is given as an example for explanation of the method according to the disclosure.

In the training phase of the system 100, the cloud database 21 acquires a plurality of training samples which are provided for example by one or more consumers via one or more of the client devices 1. As an example, one training sample received from one client device 1 may correspond to one particular male consumer who weighs 60 kg and measures 170 cm in height, and may include multiple entries of physical data related to this particular consumer, such as an entry indicating male in terms of gender, an entry indicating 60 kg in terms of body weight, and an entry indicating 170 cm in terms of body height), plural entries of nutrient intake data respectively corresponding to amounts of nutrient intake at different times of a day, and plural entries of effect data respectively corresponding to the entries of nutrient intake data. For instance, each entry of the nutrient intake data is associated with a particular amount of sugar consumed by the consumer, and the entries of the nutrient intake data correspond respectively to time points after different meals, such as two hours after lunch and two hours after dinner on a certain day. For example, the entries of the effect data are associated with effects on a particular physiological parameter, such as blood sugar level, obtained via the physiological detector 5, which may for example be implemented by a noninvasive glucose monitor based on metabolic heat and optical sensing as disclosed in European Patent No. EP1656065 B1. In this example, each of the entries of the effect data is associated with an effect on blood sugar level of the consumer at a respective one of the time points (e.g., an entry indicating that the blood sugar level is abnormal, corresponding to the entry of nutrient intake data that indicates 10 grams of sugar intake, and corresponding to the time point of two hours after lunch, and an entry indicating that the blood sugar level is normal, corresponding to the entry of nutrient intake data that indicates 1 gram of sugar intake, and corresponding to the time point of two hours after dinner). The multiple training samples may be associated with consumers different from each other in terms of bodily features. The cloud database 21 then provides the training samples to the statistical platform 22. In one implementation, for each of the training samples thus obtained, the statistical platform 22 makes a determination as to whether to delete the entry of the effect data which is directed to an abnormal blood sugar level, and the corresponding entry of the nutrient intake data which is associated with the amount of sugar which when consumed results in the abnormal blood sugar level. The determination may be made based on a criterion as shown in Table 1 below, but is not limited thereto. That is to say, only those entries of effect data that is directed to a normal blood sugar level and corresponding entries of nutrient input data will be reserved and utilized by the statistical platform 22 in building a S5 classification model 201. Thereafter, the statistical platform 22 builds the classification model 201 based on the reserved entries of nutrient intake data, the corresponding entries of effect data, and the corresponding physical data. In this way, the classification model 201 maps the physical data to the nutrient intake data associated with the amount of the sugar which when consumed results in a beneficial effect on the physiological parameter (i.e., to maintain the normal blood sugar level).

TABLE 1

| | two hours after |
|---|---|
| Condition | meal |
| Normal blood | <7.8 |
| sugar level | |
| (mmol/L) | |
| Abnormal blood | ≥7.8 |
| sugar level | |
| (mmol/L) | |

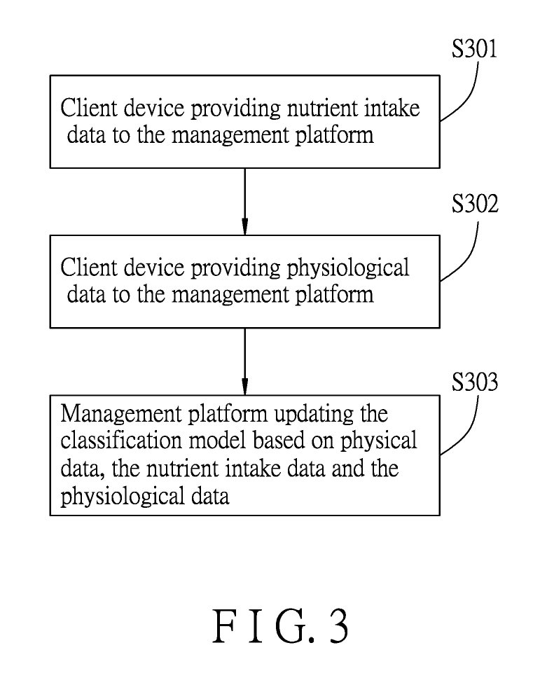

In the application phase, upon receipt from one of the client devices 1 of a to-be-classified sample that includes physical data of the male user who is 176 cm tall and weighs 75 kg and who wishes to seek the suggested amount of sugar, the management platform 23 determines suggested nutrient intake data which is associated with the suggested amount of sugar for the to-be-classified sample by inputting the physical data of the to-be-classified sample into the classification model 201 to obtain an output of the classification model 201 which serves as the suggested nutrient intake data. It should be noted that the physical data of the to-be-classified sample is associated with at least one physical feature of the user. For instance, in the current example, the to-be-classified sample may include three entries of physical data, with one indicating male in terms of gender, one indicating 176 cm in terms of body height and one indicating 75 kg in terms of body weight. The management platform 23 then provides the suggested nutrient intake data corresponding to the physical data of the to-be-classified sample to the client device 1, by referring to entries of the nutrient input data that correspond to entries of the physical data falling within the predetermined range of similarity relative to the physical data of the to-be-classified sample (for example, the entries of the physical data falling within the predetermined range of similarity relative to the physical data of male, 176 cm and 75 kg may be associated with male users between 173 cm to 179 cm in height and between 72 kg and 78 kg in weight), so that the suggested amount of sugar may be a reference for this particular user to maintain a normal blood sugar level. It should be noted that in some embodiments, the nutrient intake data may be associated with an amount of salt consumed and the physiological parameter with which the effect data is associated may be blood pressure, and this disclosure is not limited to the examples given herein. Note that while three entries of physical data are included in the to-be-classified sample, this disclosure is not limited by the number of entries of physical data included in a single to-be-classified sample; in other words, one entry may suffice in some cases. Moreover, a feedback mechanism may be implemented to update the classification model 201, and accordingly, the method of this disclosure may further include steps (S301-303) which are performed in an update phase as shown in FIG. 3 in order to update the classification model 201.

In step (S301), one of the client devices 1 (e.g., the one of the client devices 1 that had previously provided the to-be-classified sample and received the suggested nutrient intake data in return) provides plural entries of nutrient intake data to the management platform 23. Each of the entries of nutrient intake data is associated with an amount of the nutrient consumed by the user of the one of the client devices 1 as previously suggested by the suggested nutrient intake data mentioned above. The entries of nutrient intake data correspond respectively to a sequence of time points. The management platform 23 records the plural entries of nutrient intake data indexed respectively by the sequence of time points in an account corresponding to the one of the client devices 1 in the cloud database 21.

In step (S302), the one of the client devices obtains, via a corresponding one of the physiological detectors 5, plural entries of physiological data. Each of the entries of physiological data is associated with an effect on a physiological parameter of the user after a respective one of the sequence of time points at which the user consumes the amount of the nutrient as previously suggested by the suggested nutrient intake data, such as, two hours afterameal containing the suggestedamount of a particular nutrient. The one of the client devices 1 then transmits the plural entries of physiological data to the management platform 23. The management platform 23 records the plural entries of physiological data indexed respectively by the sequence of time points in the account in the cloud database 21.

In step (S303), the management platform 23 updates the classification model 201 based on the physical data of the to-be-classified sample, the plural entries of nutrient intake data and the plural entries of physiological data. As a result, the suggested nutrient intake data for a next to-be-classified sample may be more accurate.

For example, in the application phase, the management platform 23 receives, from the user via one of the client devices 1, a to-be-classified sample, the physical data of which includes body weight value of the user. The management platform 23 inputs the physical data of the to-be-classified sample into the classification model 201 so as to obtain an output of the classification model 201 which serves as the suggested nutrient intake data forthebodyweightvalueoftheto-be-classifiedsample, and transmits the suggested nutrient intake data to the user via the one of the client devices 1. When the user is, for example, satisfied with the suggested nutrient intake data, the classification model 201 does not need to be updated. On the other hand, when the user is not satisfied with the suggested nutrient intake data, the system 100 may be operated in the update phase so that the classification model 201 can be updated by the one of the client devices 1 providing plural entries of nutrient intake data and plural entries of physiological data (that correspond to the user) to the management platform 23. Each of the entries of nutrient intake data is associated with an amount of the nutrient consumed by the user as suggested by the suggested nutrient intake data, and the entries of nutrient intake data correspond respectively to a sequence of time points. Each of the entries of physiological data is associated with an effect on a physiological parameter of the user after a respective one of the sequence of time points at which the consumer consumes the suggested amount of the nutrient. The management platform 23 utilizes the physical data of the to-be-classified sample (i.e., the body weight value of the user), the entries of nutrient intake data and the entries of physiological data to serve as feedback to update the classification model 201. As a result, when the management platform 23 receives, from another user via one of the client devices 1, another to-be-classified sample, the physical data thereof including body weight value similar to that of the previously mentioned user, the output of the classification model 201 thus updated may be more appropriate for the another to-be-classified sample.

In summary, the method and the system 100 for suggesting nutrient intake according to this disclosure enable a user to input the physical data, such as body weight value, via the client device 1 which executes the application program 101. The physical data is provided to the management platform 23, and is inputted into the classification model 201 previously built so as to obtain the suggested nutrient intake data that indicates the suggested amount of the nutrient suitable for the body weight value. Moreover, if the user is not satisfied with the effect on the physiological parameter which results from consuming the suggested amount of the nutrient, the classification model 201 may be updated by feeding the body weight value, the suggested nutrient intake data and the corresponding physiological data back to the classification model 201 in an attempt to make the suggested nutrient intake data for a next user having similar body weight value more appropriate.

In the description above, for the purposes of explanation, numerous specific details have been set forth in order to provide a thorough understanding of the embodiment. It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects.

While the disclosure has been described in connection with what is considered the exemplary embodiment, it is understood that this disclosure is not limited to the disclosed embodiment but is intended to cover various arrangements included within the spirit and scope of the broadest interpretation so as to encompass all such modifications and equivalent arrangements.

What is claimed is:

1. A method for suggesting nutrient intake, to be implemented by a statistical platform, a management platform and a client device, the method comprising:
a) obtaining, by the statistical platform, a training sample that includes physical data which is associated with a physical feature of a consumer, nutrient intake data which is associated with an amount of a nutrient consumed by the consumer, and effect data which is associated with an effect on a physiological parameter of the consumer after the consumer has consumed the amount of the nutrient;
b) building, by the statistical platform, a classification model based on the physical data, the nutrient intake data and the effect data of the training sample thus obtained, and providing, by the statistical platform, the classification model to the management platform, the classification model mapping the physical data to the nutrient intake data associated with the amount of the nutrient which when consumed results in the effect on the physiological parameter; and
c) determining, by the management platform when receiving a to-be-classified sample that includes physical data from the client device, suggested nutrient intake data which is associated with a suggested amount of the nutrient for the to-be-classified sample by inputting the physical data of the to-be-classified sample into the classification model to obtain an output of the classification model which serves as the suggested nutrient intake data.

2. The method as claimed in claim 1, to be further implemented by a physiological detector cooperating with the client device, the method further comprising:
d) providing, by the client device to the management platform, plural entries of nutrient intake data each of which is associated with an amount of the nutrient consumed by a user of the client device as suggested by the suggested nutrient intake data, and which correspond respectively to a sequence of time points;
e) obtaining, by the client device via the physiological detector, plural entries of physiological data each of which is associated with a physiological response of the user after a respective one of the sequence of time points at which the user consumes the amount of the nutrient as suggested by the suggested nutrient intake data, and transmitting, by the client device, the plural entries of physiological data to the management platform; and
f) updating, by the management platform, the classification model based on the physical data of the to-be-classified sample, the plural entries of nutrient intake data and the plural entries of physiological data.

3. The method as claimed in claim 2, to be further implemented by a cloud database, and further comprising:
g) recording, by the management platform, the plural entries of nutrient intake data indexed respectively by the sequence of time points in an account corresponding to the client device in the cloud database; and
h) recording, by the management platform, the plural entries of physiological data indexed respectively by the sequence of time points in the account in the cloud database.

4. The method as claimed in claim 1, wherein the classification model is implemented by vector space model (VSM) and similarity measurement so as to realize statistical classification, and is utilized to determine the suggested nutrient intake data for the to-be-classified sample.

5. A system for suggesting nutrient intake, adapted to cooperate with a client device, said system comprising:
a statistical platform configured to
obtain a training sample that includes physical data which is associated with a physical feature of a consumer, nutrient intake data which is associated with an amount of a nutrient consumed by the consumer, and effect data which is associated with an effect on a physiological parameter of the consumer after the consumer has consumed the amount of the nutrient,
build a classification model based on the physical data, the nutrient intake data and the effect data of the training sample thus obtained, and
provide the classification model that maps the physical data to the nutrient intake data associated with the amount of the nutrient which when consumed results in the effect on the physiological parameter; and
a management platform configured to
obtain the classification model, and
determine, when receiving a to-be-classified sample that includes physical data from the client device, suggested nutrient intake data which is associated with a suggested amount of the nutrient for the to-be-classified sample by inputting the physical data of the to-be-classified sample into the classification model to obtain an output of the classification model which serves as the suggested nutrient intake data.

6. The system as claimed in claim 5, the client device cooperating with a physiological detector, wherein:
said management platform is further configured to
obtain from the client device, plural entries of nutrient intake data each of which is associated with an amount of the nutrient consumed by a user of the client device as suggested by the suggested nutrient intake data, and which correspond respectively to a sequence of time points,
obtain from the client device, plural entries of physiological data each of which is associated with a physiological response of the user after a respective one of the sequence of time points at which the user consumes the amount of the nutrient as suggested by the suggested nutrient intake data, and each of which is obtained by the client device via the physiological detector, and
update the classification model according to the physical data of the to-be-classified sample, the plural entries of nutrient intake data and the plural entries of physiological data.

7. The system as claimed in claim 6, wherein:
said management platform is configured to
record the plural entries of nutrient intake data indexed respectively by the sequence of time points in an account corresponding to the client device in a cloud database, and
record the plural entries of physiological data indexed respectively by the sequence of time points in the account in the cloud database.

8. The system as claimed in claim 5, wherein the classification model is implemented by vector space model (VSM) and similarity measurement so as to realize statistical classification and to determine the suggested nutrient intake data for the to-be-classified sample.
